(19) United States
(12) Patent Application Publication
(10) Pub. No.: US 2023/0161057 A1
(43) Pub. Date: May 25, 2023

(54) PEROVSKITE-BASED X-RAY IMAGE DETECTOR

(71) Applicant: University of Saskatchewan, Saskatoon (CA)

(72) Inventors: Timothy Lewis Kelly, Saskatoon (CA); Yunlong Li, Saskatoon (CA); Richard Pettipas, Saskatoon (CA)

(21) Appl. No.: 17/908,722

(22) PCT Filed: Mar. 4, 2021
(86) PCT No.: PCT/CA2021/050289

§ 371 Date: Sep. 1, 2022

Related U.S. Application Data
(60) Provisional application No. 62/985,616, filed on Mar. 5, 2020.

(51) Int. Cl. G01T 1/24; C09D 11/52; C09D 11/037; C09D 11/033; C09D 11/106

(52) U.S. Cl. CPC G01T 1/24; C09D 11/52; C09D 11/037; C09D 11/033; C09D 11/106

(57) ABSTRACT

A direct conversion x-ray detection apparatus having a planar x-ray detection layer having a detection layer upper surface and a detection layer lower surface, the planar x-ray detection layer including a lead halide perovskite material; a top electrode layer above the detection layer upper surface; a bottom electrode layer below the detection layer lower surface and in conductive communication with the top electrode layer through the x-ray detection layer to apply a bias voltage across the x-ray detection layer; and a blocking layer between the x-ray detection layer and the top electrode layer to inhibit a dark current, the blocking layer including a polymer selected from the group comprising polyacrylates, polyimides, polyamides, polysulfones, polystyrenes, and polycarbonates.

### FIELD

Embodiments described herein relate to image detectors, and in particular to perovskite-based x-ray image detectors.

### BACKGROUND

U.S. Pat. No. 10,199,588 (Belcher et al.) purports to disclose that a planar mixed-metal perovskite solar cell can exhibit many favorable properties including high efficiencies and tunable electronic properties. Belcher et al. further purports to discloses that the incorporation of different metal species (i.e., Co, Cu, Fe, Mg, Mn, Ni, Sn, Sr, and Zn) into the film is made possible by the solubility of either each metal's divalent acetate or halide compound in a solvent.

U.S. Pat. No. 9,520,512 (Irwin et al.) purports to disclose that photovoltaic devices such as solar cells, hybrid solar cell-batteries, and other such devices may include an active layer disposed between two electrodes. Irwin et al. further purports to disclose that the active layer may have perovskite material and other material such as mesoporous material, interfacial layers, thin-coat interfacial layers, and combinations thereof. Irwin et al. further purports to disclose that the perovskite material may be photoactive. Irwin et al. further purports to disclose that the active layer may include a titanate. Irwin et al. further purports to disclose that the perovskite material may be disposed between two or more other materials in the photovoltaic device. Irwin et al. further purports to disclose that inclusion of these materials in various arrangements within an active layer of a photovoltaic device may improve device performance. Irwin et al. further purports to disclose that other materials may be included to further improve device performance, such as, for example: additional perovskites, and additional interfacial layers.

US 2019 0221748 (Wudl et al.) purports to disclose sulfur-fused perylene diimides (PDIs) having the formula 2PDI-nS, wherein n is an integer. Wudl et al. further purports to disclose that such sulfur-fused PDIs (e.g., 2PDI-2S, 2PDI-3S, and 2PDI-4S) are incorporated as electron acceptors in an active region of a bulk heterojunction solar cell and/or as an electron transport layer. Wudl et al. further purports to disclose that example solar cells exhibit a power conversion efficiency above 5% and a fill factor above 70% (a record high for non-fullerene bulk heterojunction solar cell devices) when 2PDI-nS is used as the electron acceptor. Wudl et al. further purports to disclose that in addition, the solar cells exhibit low open circuit voltage (Voc) loss.

US 2019 0051830 (Druffel et al.) purports to disclose a perovskite thin film and method of forming a perovskite thin film. Druffel et al. purports to disclose that the perovskite thin film includes a substrate, a hole blocking/electron transport layer, and a sintered perovskite layer. Druffel et al. purports to disclose that the method of forming the perovskite solar cell includes depositing a perovskite layer onto a substrate and processing (for example, by sintering) the perovskite layer with intense pulsed light to initiate a radiative thermal response that is enabled by an alkyl halide additive.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

According to one broad aspect of the teachings described herein, which may be used alone or in combination with any other aspect or aspects, a direct conversion x-ray detection apparatus, comprising a planar x-ray detection layer having a detection layer upper surface and a detection layer lower surface, the planar x-ray detection layer including a lead halide perovskite material; a top electrode layer above the detection layer upper surface; a bottom electrode layer below the detection layer lower surface and in conductive communication with the top electrode layer through the x-ray detection layer to apply a bias voltage across the x-ray detection layer; and a blocking layer between the x-ray detection layer and the top electrode layer to inhibit a dark current, the blocking layer including a polymer selected from the group comprising polyacrylates, polyimides, polyamides, polysulfones, polystyrenes, and polycarbonates.

In some embodiments, the planar x-ray detection layer has a thickness between the detection layer upper surface and the detection layer lower surface of at least 100 micrometers.

In some embodiments, the thickness is between 400 micrometers and 1500 micrometers.

In some embodiments, the blocking layer includes poly(methyl methacrylate).

In some embodiments, the lead halide perovskite material is MAPbI₃.

In some embodiments, the bottom electrode includes a thin-film transistor array.

In some embodiments, the bottom electrode includes a transparent conducting material.

In some embodiments, the transparent conducting material includes at least one of indium tin oxide (ITO) and fluorine doped tin oxide (FTO).

In some embodiments, the top electrode includes at least one of silver and gold.

In some embodiments, the blocking layer includes a hole transport layer inhibiting electron transport.

In some embodiments, the x-ray detection apparatus further comprises an electron transport layer between the bottom electrode and the planar x-ray detection layer, the electron transport layer inhibiting hole transport.

In accordance with an aspect of the present disclosure, which may be used alone or in combination with any other aspect or aspects, a detection layer may be made using an ink and/or by printing and/or by coating.

In an aspect of the present disclosure, which may be used alone or in combination with any other aspect or aspects, there may be a solution-based ink for use in forming a detection layer. In an aspect of the present disclosure, which may be used alone or in combination with any other aspect or aspects, there may be a pigment-based ink for use in forming a detection layer.

### DRAWINGS

FIG. 1 is a schematic cross sectional view of an x-ray detector, in accordance with an embodiment;

FIG. 2 is a schematic perspective view of a perovskite material, in accordance with an embodiment;

FIG. 3 is a graph of calculated band structure for a cubic perovskite phase of MAPbI₃;

FIG. 4 is a schematic perspective view of the relationship between the Brillouin zones associated with the cubic (outer cuboid) and orthorhombic (inner cuboid) crystalline phases of MAPbI₃;

FIG. 5 is a graph of calculated x-ray absorption for various materials;

FIG. 6 is a graph comparing the calculated mass attenuation coefficient of various materials;

FIG. 7 is a graph of x-ray response for a tested x-ray detector;

FIG. 8 is a J-V curve graph;

FIG. 9 is a time of flight measurement graph;

FIG. 10 is a graph comparing the dark current density of MAPbI₃ to that of a-Se;

FIG. 11 is a graph comparing the sensitivity of MAPbI₃ to that of a-Se;

FIG. 12 is a graph comparing the current density of MAPbI₃ to that of a-Se;

FIG. 13 is a graph comparing the charge collected from a MAPbI₃-based detector to that of a-Se;

FIG. 14 is a schematic perspective view of an x-ray detector, in accordance with an embodiment;

FIG. 15 is a schematic perspective view of an x-ray detector, in accordance with an embodiment;

FIG. 16 is a J-V Curve graph of the x-ray detector of FIG. 23;

FIG. 17 shows a scotch tape adhesion test of example detection layers;

FIG. 18 shows SEM Morphologies of example layers;

FIG. 19 shows example detection layers after drying;

FIG. 20 is a schematic diagram of an example printing operation; and

FIG. 21 is a schematic diagram of an example drying operation.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors or owners do not intend to abandon, disclaim or dedicate to the public any such invention by its disclosure in this document.

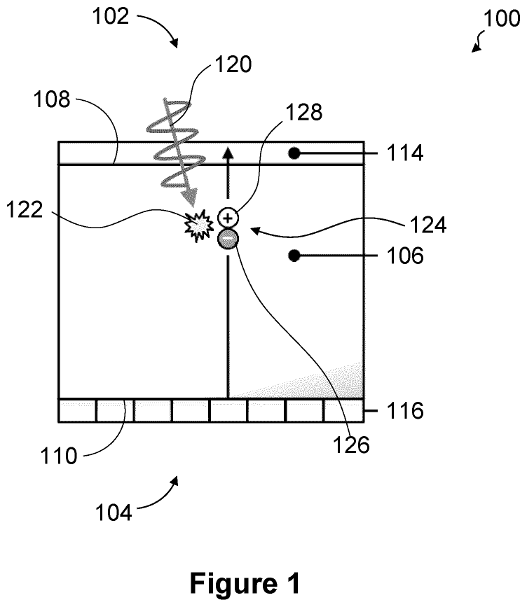

Referring to FIG. 1, depicted is an example x-ray detector 100 having an upper side 102 and a lower side 104. The illustrated example X-ray detector 100 is a direct conversion detector, although aspects of the present disclosure may be used with indirect conversion detectors in some examples. In some examples, direct conversion detectors have greater special resolution than indirect conversion detectors.

The illustrated example x-ray detector 100 of FIG. 1 includes a planar x-ray detection layer 106 having a detection layer upper surface 108 and a detection layer lower surface 110. The example x-ray detector 100 also has a top electrode 114 above the detection layer upper surface 108 and a bottom electrode 116 below the detection layer lower surface 110. In the depicted example, the top and bottom electrodes 114, 116 are each immediately adjacent the detection layer 106, however in other embodiments one or more layers may be between.

During an imaging session, x-ray photons may be incident on detector 100. In the illustrated example, x-ray photons are incident on the upper side 102 of detector 100. An x-ray photon 120 may enter detector 100 and be absorbed by the detection layer 106 at a detection site 122. When an x-ray photon 120 is absorbed by detection layer 106 a pair of charge carriers 124 may be generated.

Top and bottom electrodes 114, 116 are in conductive communication through the x-ray detection layer 106, and a bias voltage may be applied during an imaging session. When charge carriers 124 are generated, a bias voltage may urge each charge carrier in a predetermined direction. In the illustrated example, a bias voltage may urge an electron 126 towards bottom electrode 116 and electron hole 128 towards top electrode 114.

In some embodiments, one of top and bottom electrodes 114, 116 may be pixelated so that a charge carrier may be used to create an x-ray image. In the illustrated example, bottom electrode 116 is a pixilated electrode. In some embodiments, a bottom electrode may be a thin-film transistor (TFT), although in other embodiments an electrode may include a complementary metal-oxide-semiconductor (CMOS) or charge-coupled device (CCD) as well or instead.

In some embodiments, a bottom electrode may include a transparent material such as indium tin oxide (ITO) and/or fluorine doped tin oxide (FTO), and in some embodiments a TFT may be formed on a transparent conducting material. The wetting of an ink on the conductive metal oxide such as ITO and FTO may be favorable. Silver (Ag) or Gold (Au) may also be used in an electrode in some embodiments. In some embodiments, one electrode, such as a bottom electrode 116, is a TFT formed on a transparent conducting material while the other is a silver or gold electrode.

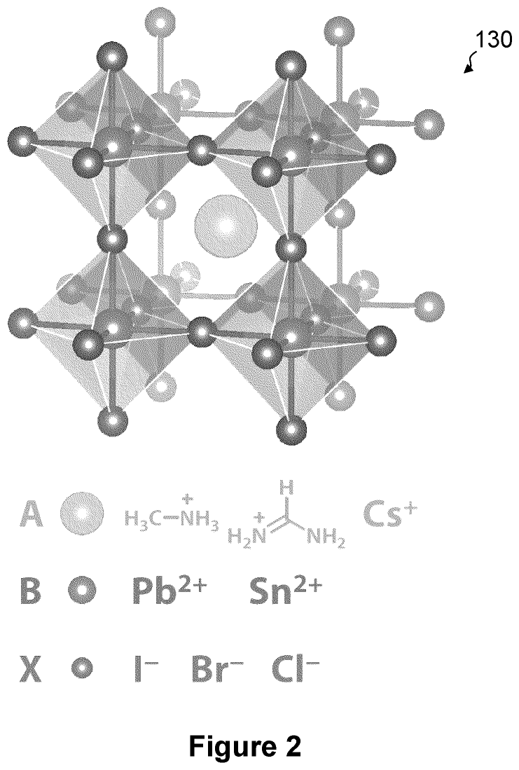

Referring now to FIG. 2, in some examples perovskite materials 130 have been found to be sensitive to x-rays. Perovskites are crystal structures with the general formula ABX₃. In some examples, A=Cs⁺, MA⁺ or FA⁺, B=Pb²⁺ or Sn²⁺, and X=I⁻, Br⁻ or Cl⁻. In some examples, a dense perovskite such as a lead halide perovskite may be used. The density, high atomic number of Pb and I, may lead to high x-ray attenuation coefficients in some examples. These particular semiconductors may have good carrier transport properties in some examples. In some examples, a methylammonium perovskite of chemical formula MAPbI₃ may be used. MAPbI₃ may have reasonable film-forming properties in some examples. In some examples, MAPbBr3 tends to crystallize rapidly into big cubic crystals that don't pack together very well. In some examples, some mixed-ion compositions may be used.

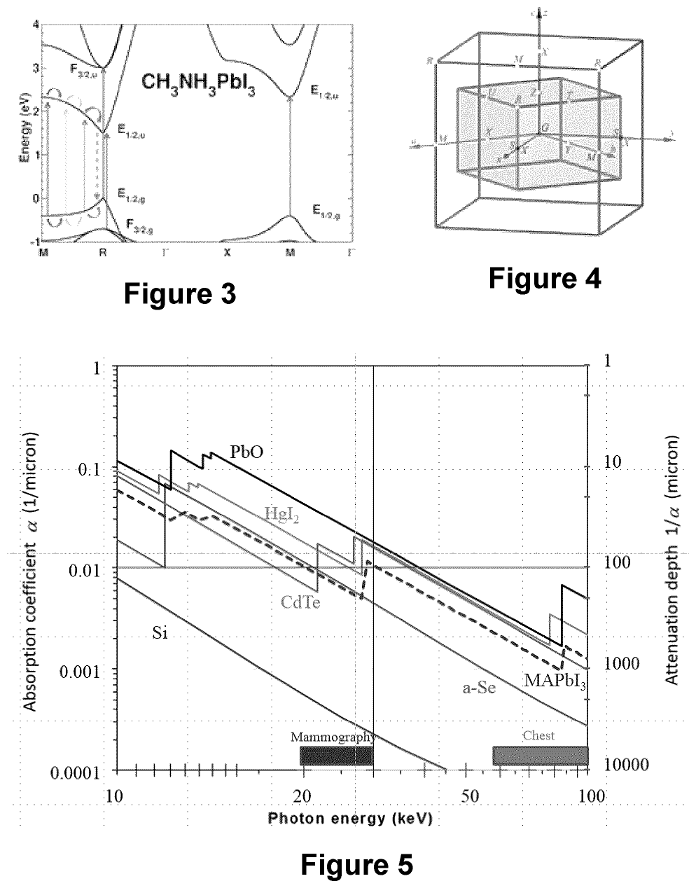

FIG. 3 shows the calculated band structure of MAPbI₃, showing energy versus wavevector for points and lines of symmetry within the first Brillouin zone for the cubic perovskite phase of MAPbI₃. FIG. 4 shows the relationship between the Brillouin zones associated with the cubit (outer cuboid) and orthorhombic (inner cuboid) crystalline phases.

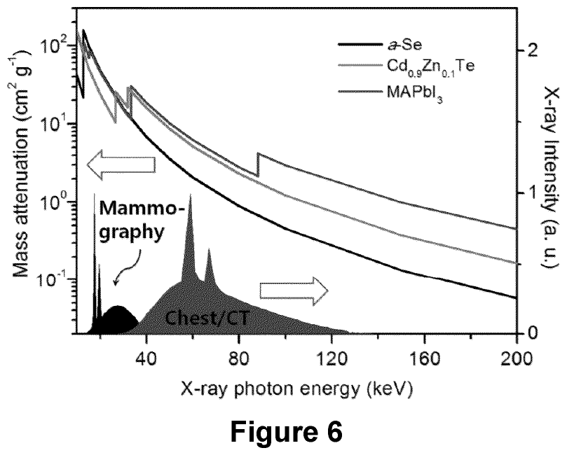

Amorphous selenium (a-Se) has been suggested for use in forming a detector layer of an x-ray detector. FIGS. 5 and 6 are graphs comparing calculated absorption coefficient and mass attenuation of uncoated MAPbI₃ perovskite to other materials, including a-Se. As indicated in the graphs, the absorption coefficient and mass attenuation of MAPbI₃ perovskite is similar to that of a-Se in the mammography region, and better than that of a-Se in the chest radiography region.

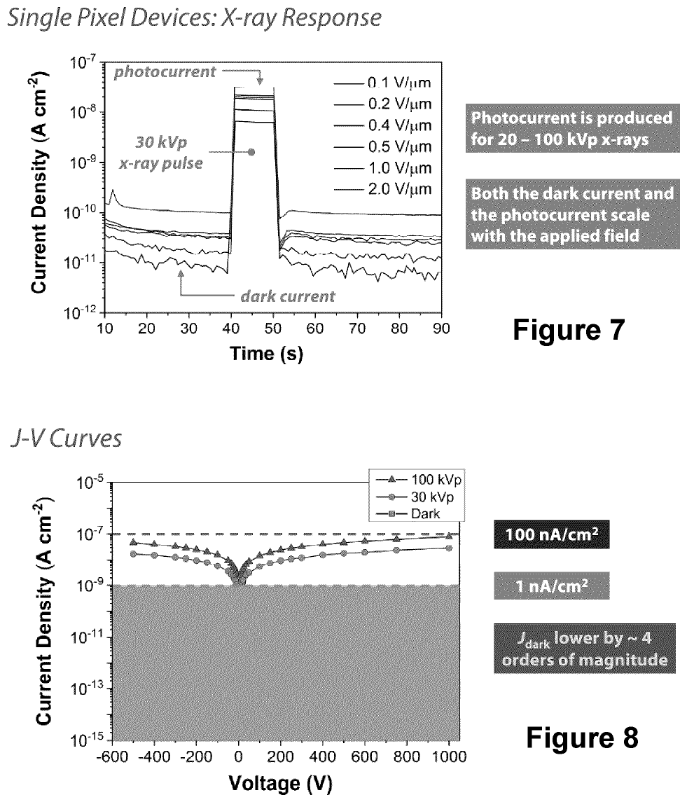

Referring now to FIG. 7, measurements may be made in some examples by applying a bias voltage, measuring current density, and then applying an x-ray pulse of ˜10 s duration. The current increases; then the pulse is turned off, and the current returns to baseline. In the illustrated example there is a large (many orders of magnitude) difference between the photocurrent and the dark current. The illustrated example is of a MAPbI₃ detector with a PMMA blocking layer.

Referring now to FIG. 8, illustrated is a dark J-V curve for the example device used for FIG. 7. The shaded portion shows an example of what may be an acceptable level in a commercial product.

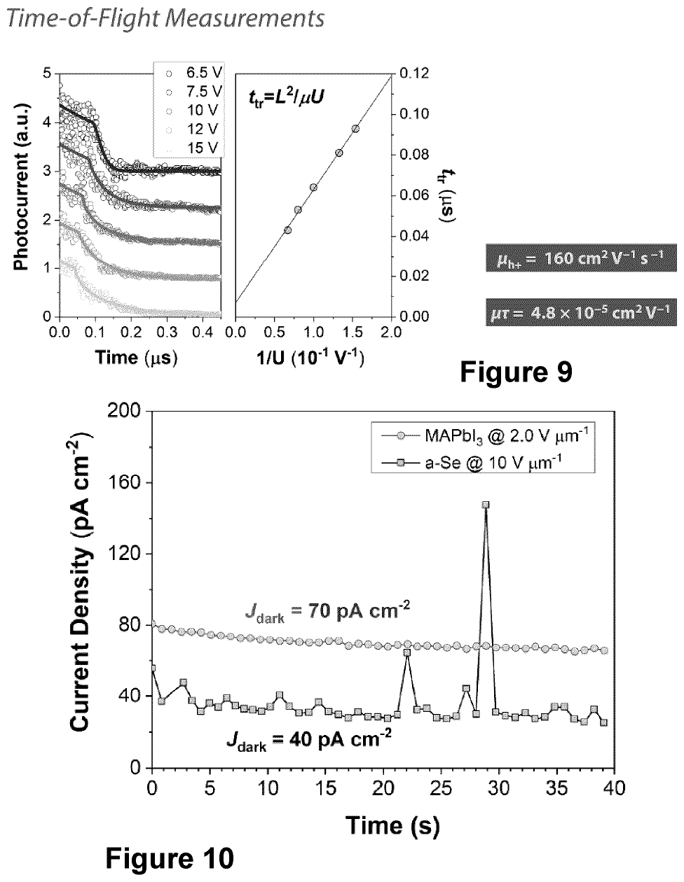

Referring now to FIG. 9, the illustrated example are ToF measurements for a material as in FIGS. 7 and 8 but without the PMMA blocking layer. The ToF measurements probe how long it takes carriers to get from one side of the device to the other for calculating the charge carrier mobility and trapping time.

Referring now to FIG. 10, the illustrated example measurements are from a device as in FIGS. 7 and 8. In the illustrated example, measurement of the dark current, shows it is competitive with commercial a-Se technology.

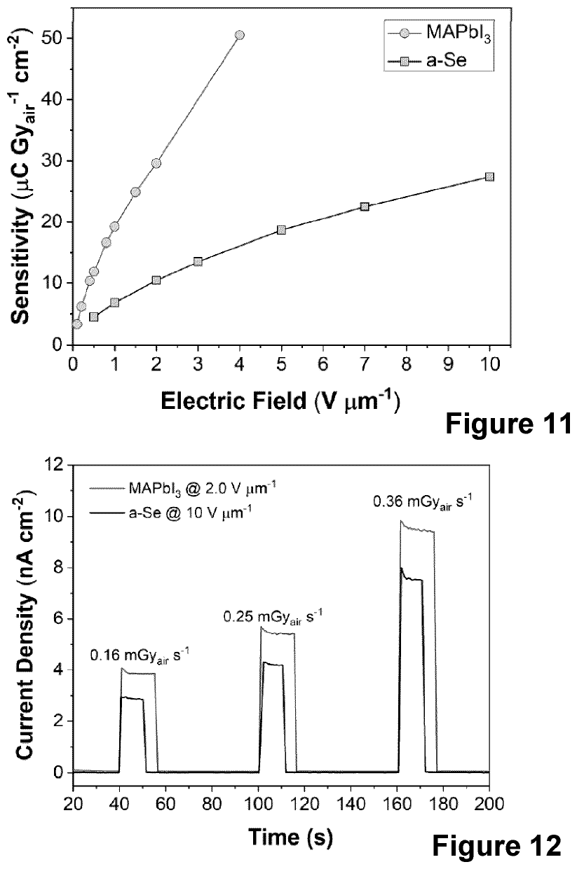

Referring now to FIG. 11, the illustrated example measurements are from a device as in FIGS. 7 and 8. In the illustrated example, sensitivity vs. applied field show that the example device is able to outperform some commercial devices.

Referring now to FIG. 12, the illustrated example measurements are from a device as in FIGS. 7 and 8. In the illustrated example, photocurrent scales with the absorbed dose.

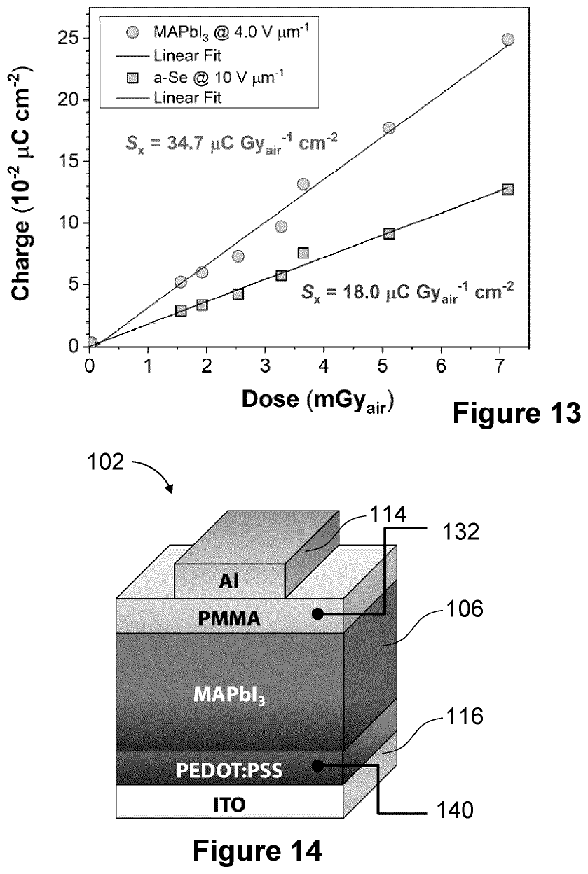

Referring now to FIG. 13, the illustrated example measurements are from a device as in FIGS. 7 and 8. In the illustrated example, a more rigorous assessment of the x-ray sensitivity is shown, including many measurements at different dose rates and a linear regression.

In accordance with an aspect of the present disclosure, which may be used alone or in combination with any other aspect or aspects, an x-ray detection apparatus may include a blocking layer between a lead halide perovskite x-ray detection layer and an electrode to inhibit dark current and produce an image with reduced signal noise. A dark current is the current that is collected in the absence of any x-ray illumination. It may be caused in some examples by either charge injection (given a high applied bias) or thermal generation (promotion across the band gap by thermal energy).

Blocking layers may be used in some examples to reduce this dark current. In some examples, steady-state current generation requires a complete circuit, so in some examples a blocking layer between a detection layer and an electrode on either side of the blocking layer can be effective. In some examples a blocking layer is placed between a detection layer and a negative electrode.

In some examples, a blocking layer formed of and/or including a polymer selected from the group comprising polyacrylates, polyimides, polyamides, polysulfones, polystyrenes, and polycarbonates may assist in selectively inhibiting dark current (i.e., inhibiting dark current to a greater degree than signal current). In some examples perylene diimide (PDI) may be used. In some examples, a blocking layer formed of and/or including poly(methyl methacrylate) (PMMA) may be particularly effective at selectively inhibiting dark current.

In some examples, a detector device may incorporate a hole transport layer 140 or an electron transport layer. In some examples a hole transport layer 140 and/or an electron transport layer may improve the performance of a detector 100. Hole and electron transport layer may inhibit the movement of electrons and holes, respective, in a region adjacent an electrode. In some examples, a hole transport layer may be between the detector layer 106 and a top electrode 114 and an electron transport layer may be between the detector layer 106 and a bottom electrode 116. Positioning of the electron transport layer and hole transport layer in examples which include one or both of these layers may depend on the polarity of the electrodes. In some examples a blocking layer may be included in a hole or electron transport layer.

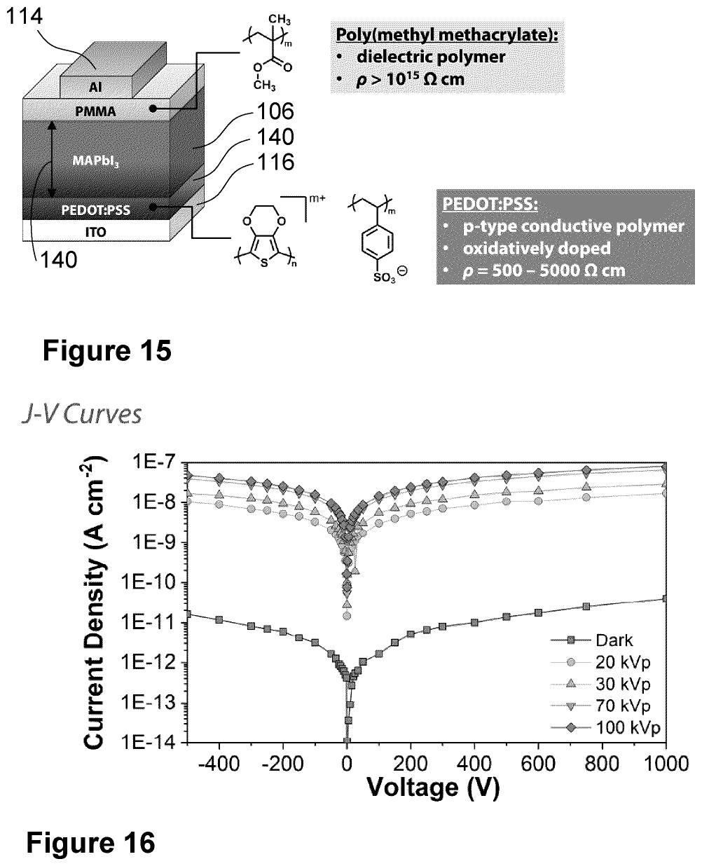

Referring now to FIGS. 14 to 16, in some examples, a blocking layer 132 is formed of PMMA and a hole transport layer 140 may be formed of poly(3,4-ethylenedioxythiophene) polystyrene sulfonate (PEDOT:PSS). In some examples, a blocking layer may be a hole or electron transport layer or may be instead of a hole or electron transport layer. In the illustrated example, the blocking layer 132 is instead of an electron transport layer. In some examples, a blocking layer 132 may be used without either a hole or electron transport layer. As shown in the graph of FIG. 16, the x-ray detector of FIGS. 14 and 15 may have a significantly improved ratio of signal current to dark current. In the example of FIG. 16, shown is the J-V curves of a MAPbI₃/PMMA device in the dark and under x-ray irradiation. The dark current is very low, even at relatively high applied bias.

Referring again to FIGS. 14 and 15, the thickness 140 of an x-ray detection layer 106 may affect the proportion of x-ray radiation that is absorbed. Unabsorbed x-ray radiation may be wasted with respect to imaging, and may also be damaging to nearby individuals. In some examples, an x-ray detection layer may absorb all or substantially all the x-ray radiation incident on the x-ray detection layer. In some examples, the thickness is much greater than the penetration depth of the expected x-ray radiation. In some examples in which the x-ray detection layer includes a lead halide perovskite layer, the lead halide perovskite layer may be at least 100 micrometers thick.

Various applications may require the use of various x-ray energies, and higher x-ray energies may require the use of thicker x-ray detection layers to reduce the amount of waste radiation. In some examples, a detector for use with 10 to 30 kiloelectron volt x-rays, such as x-rays for mammogram imaging, may have a lead halide perovskite detector layer that is at least 100 micrometers thick. In some examples, a lead halide perovskite detector layer for detecting 10 to 30 kiloelectron volt x-rays may be at least 200 micrometers thick. In some examples, a lead halide perovskite detector layer for use with 70 to 100 kiloelectron volt x-rays, such as x-rays for imaging a chest of an individual, may be at least 300 micrometers thick. In some examples, a lead halide perovskite detector layer for detecting 70 to 100 kiloelectron volt x-rays may be between 400 and 1500 micrometers.

In some examples, additional layers or coatings may be applied. For example, a moisture coating may be applied to protect the detection device 100 from moisture. In some examples, a moisture coating may be applied over the entire device 100 without passing between a detection layer 106 and an electrode 114, 116, since a barrier between a detection layer 106 and an electrode 114, 116 may inhibit signal current.

In accordance with an aspect of the present disclosure, which may be used alone or in combination with any other aspect or aspects, a detection layer may be made using an ink and/or by printing and/or coating. Printing or coating of a material may allow for accurate deposition of a layer of material, and may be faster and/or cheaper than many other manufacturing techniques such as thermal evaporation. In an aspect of the present disclosure, which may be used alone or in combination with any other aspect or aspects, there may be a solution-based ink for use in forming a detection layer. In an aspect of the present disclosure, which may be used alone or in combination with any other aspect or aspects, there may be a pigment-based ink for use in forming a detection layer.

A layer of a detector apparatus 100 may be formed using various techniques. In some examples, an ink formulation may be used to form a layer, such as to form a perovskite detection layer 106. Example ink application techniques include dropcasting, spincoating, brush painting, inkjet printing, stamp printing, spray coating, dipcoating, zone casting, hollow pen writing, blade coating, slot die coating, or solution shearing.

Factors such as ink concentration, flow rate, and substrate affect film thickness.

As discussed above, thickness of a layer, particularly a detection layer 106, may affect the performance of a detection apparatus 100. Further, forming a detection layer 106 in multiple printing passes may produce a layer of lower quality than formed by a single printing pass. It has been found that in some examples a detection layer of 200 micrometers formed in two passes is only marginally more effective than a detection layer of 100 micrometers formed in a single pass. Accordingly, ink formulations may be used which allow a sufficiently thick perovskite detection layer to be formed in as few printing passes as possible, preferably in a single printing pass. A perovskite detection layer printed in one or few passes may be dense and of high quality, with few grain boundaries.

Ink formulation may determine factors such as the viscosity and drying time of an ink. Ink formulation may also determine how well a detection layer formed from an ink adheres to a substrate. It has been found that thin perovskite detection layers, such as layers in the range of a few hundred nanometers found in solar cells, adhere better to underlying layers than the thicknesses needed for x-ray detection. Without the necessary additives, a perovskite layer of 100 micrometers or more may peel or delaminate from a substrate.

In one example, the ink formulation is a solution-based ink formulation. In another example, the solution-based ink formulation comprises one or more perovskite precursors, a solvent or mixture of solvents, a binder and an additive. A solution-based ink may be, in some examples, a yellow ink, used to create a black detection layer once set.

In one embodiment, the perovskite precursor is a methylammonium perovskite of chemical formula MAPbI₃. In one embodiment, the perovskite precursor is present in the formulation at a concentration of between about 1.0 M and about 5.0 M, optionally about 3.5 M. In one embodiment, the perovskite is formed from lead iodide (PbI₂) and methylammonium iodide CH₃—NH₃⁺I⁻ precursors, to form MAPbI₃.

In another embodiment, the solvent is n-methyl pyrrolidone (NMP) or dimethyl formamide (DMF), or a combination thereof. In another embodiment, the solvent is a mixture of n-methyl pyrrolidone (NMP) or dimethyl formamide (DMF), optionally in a ratio of between about 1:2 to about 2:1 (w/w) (NMP:DMF), or about 1:1. Examples of other solvents include DMSO and gamma-butyrolactone (GBL).

In another embodiment, the solution-based ink formulation comprises a binder which is polyvinyl pyrrolidone (PVP), or derivatives thereof. In one embodiment, the PVP has the structure:

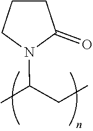

where n is an integer between 2 and 200,000.

In another embodiment, the binder is present in the formulation in an amount between about 1% to about 10% (w/w), or about 5% (w/w). Other binders and/or other additives may be used in addition or in alternative in some examples.

In another embodiment, the solution-based ink formulation comprises an additive which is a quarternary ammonium salt. In another embodiment, the quarternary ammonium salt is cetyltrimethyl ammonium bromide (CTAB). In one embodiment, the additive is present in the formulation in an amount between about 1% and about 5% (w/w), or about 2% (w/w).

In another embodiment, the solution-based ink formulation comprises:
a) lead iodide (PbI₂) and methylammonium iodide CH₃—NH₃⁺I⁻;
b) a solvent comprising a combination of NMP and DMF;
c) PVP; and
d) CTAB.

In another embodiment, the solution-based ink formulation comprises:
a) lead iodide (PbI₂) and methylammonium iodide CH₃—NH₃⁺I⁻ at a concentration of about 3.5 M;
b) a solvent comprising a combination of NMP and DMF at a ratio of about 1:1 (w/w);
c) PVP present in an amount of about 5.0% (w/w); and
d) CTAB present in an amount of about 2.0% (w/w).

This may give a film of usable quality in some examples, with a viscosity low enough to be slot-die coated. In some examples, slot die coating is preferable to blade coating.

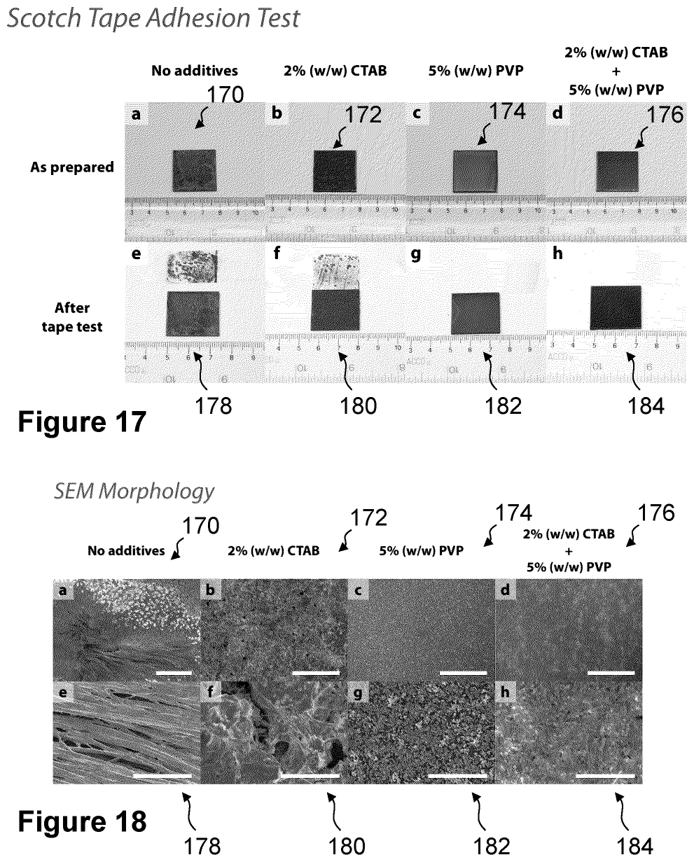

Referring to FIG. 17, in some examples one or more binders and/or additives may be added to increase adhesion. For example, addition of one or more binders and/or additives may improve the performance of an ink layer after it has been permitted to dry for a period of time in a tape test, in which a layer of tape is applied to an exposed surface of a dried ink layer and removed to show whether one or more portions of the ink layer are removed by the tape.

In the illustrated example of FIG. 17, four ink formulations formed from lead iodide (PbI₂) and methylammonium iodide CH₃—NH₃⁺I⁻ at a concentration of about 3.5 M and a solvent comprising a combination of NMP and DMF at a ratio of about 1:1 (w/w) were formed. A first formulation contained no PVP and no CTAB, a second formulation contained 2.0% (w/w) CTAB and no PVP, a third formulation contained 5% (w/w) PVP and no CTAB, and a fourth formulation contained PVP present in an amount of about 5.0% (w/w), and CTAB present in an amount of about 2.0% (w/w). Each formulation was deposited on a substrate and allowed to dry for at least 12 hours. After this drying, a first formulation formed a first example detection layer 170, a first formulation formed a first example detection layer 170, a first formulation formed a first example detection layer 170, and a first formulation formed a first example detection layer 170. To each example detection layer 170, 172, 174, 176 a piece of tape was applied and then removed to produce a tested layer 178, 180, 182, 184. As may be seen from FIG. 17, one or more additives may produce a detection layer with greater adhesion. If a film lacks sufficient adhesion, in some examples it may delaminate during fabrication, shipping, receiving, operation, etc. In some examples, passing this simple test may be used as an indicator of commercial potential.

Referring now to FIG. 18, SEM Morphologies of layers 170, 172, 174, 176, 178, 180, 182, 184 are shown. In some examples, films without an additive and/or binder are of low quality, and contain large gaps and voids. In some examples, a uniform, dense film is better from both an x-ray absorption and charge transport perspective.

In some embodiments of the disclosure, there are pigment-based ink formulations. In one embodiment, the pigment-based ink formulations comprise a perovskite precursor, a binder and a solvent. In one embodiment, pigment-based ink formulations are not limited by solubility and may have a higher concentration of perovskite precursors than a solution-based ink.

In one embodiment, the pigment-based ink formulation comprises a perovskite precursor which is a methylammonium perovskite of chemical formula MAPbI₃. In one embodiment, the perovskite precursor is present in the formulation at a concentration of between about 5.0 M and about 10.0 M, optionally about 6 M. In one embodiment, the perovskite is formed from lead iodide (PbI₂) and methylammonium iodide CH₃—NH₃⁺I⁻ to form MAPbI₃. A pigment based ink may have MAPbI₃ formed in the ink, rather than precursors. A pigment based ink may be the same or similar color as the resulting perovskite film. A pigment based ink may be black in some examples.

In one embodiment, the pigment-based ink formulation comprises a binder which is a polymer having the structural formula

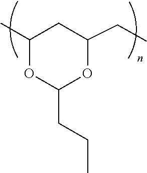

wherein n is an integer between 2 and 200,000.

In another embodiment, the binder is present in an amount between about 1.0% and about 5.0% (w/w), or optionally about 1.5% (w/w). In some examples, other binders and/or additives may be used in addition or alternative to PVB. In some examples, PVP may be used as a binder.

In another embodiment, the pigment-based ink formulation comprises a solvent which is ethanol, water or a combination thereof. In another embodiment, the solvent is a combination of ethanol and water. In a further embodiment, the ethanol and water are present in a ratio of about 60:40 to about 90:10, or about 70:30 to about 90:10, or about 85:15. Methanol and isopropanol may be used as solvents in some examples.

In another embodiment, the pigment-based ink formulation comprises:
a) MAPbI₃;
b) a solvent comprising a combination of ethanol and water; and
c) a binder having the structural formula

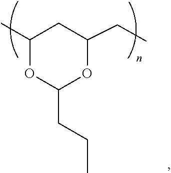

wherein n is an integer between 2 and 200,000.

In another embodiment, the solution-based ink formulation comprises:
a) MAPbI₃ at a concentration of about 6.0 M;
b) a solvent comprising a combination of ethanol and water at a ratio of about 85:15 (w/w); and
c) a binder having the structural formula

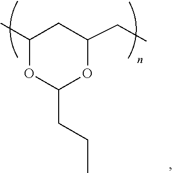

wherein n is an integer between 2 and 200,000, present at an amount of about 1.5% (w/w).

In some examples, a pigment formulation can produce much thicker films than a solution formulation in a single pass (500 μm or more) that still display good substrate adhesion.

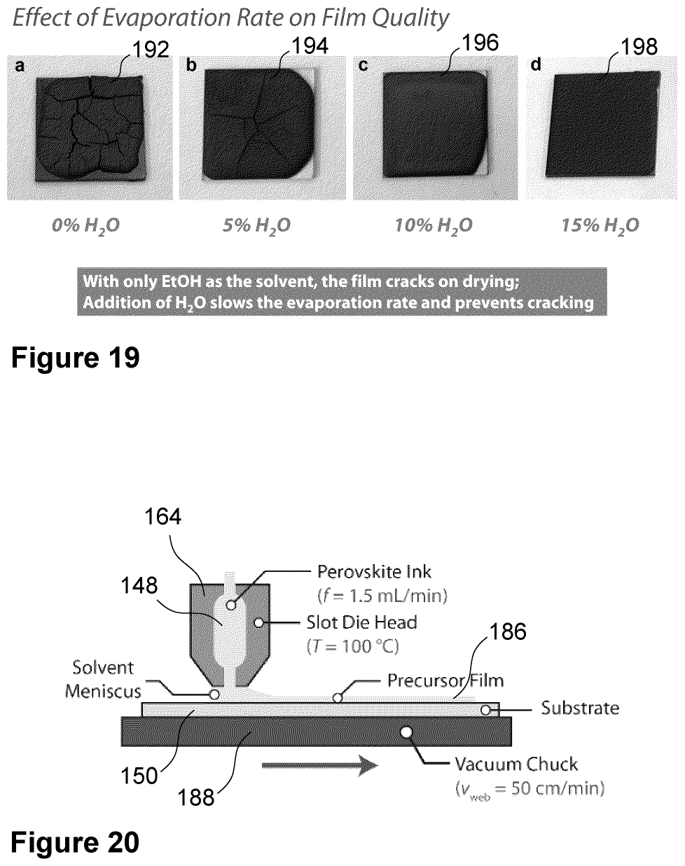

Referring now to FIG. 19, in some examples the quantity of water in a solvent may be varied. Varying quantities of water in a solvent of an ink may affect evaporation rate and film quality.

In the illustrated example of FIG. 19, four ink formulations were formed from lead iodide (PbI₂) and methylammonium iodide CH₃—NH₃⁺I⁻ at a concentration of about 6.0 M and a binder having the structural formula

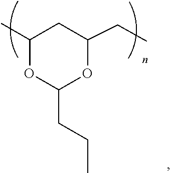

wherein n is an integer between 2 and 200,000, present at an amount of about 1.5% (w/w).

A first formulation included a solvent containing ethanol and no water and was applied to form detection layer 192, a second formulation included a solvent containing ethanol and water at a ratio of about 95:5 (w/w) and was applied to form detection layer 194, a third formulation included a solvent containing ethanol and water at a ratio of about 90:10 (w/w) and was applied to form detection layer 196, and a fourth formulation included a solvent containing ethanol and water at a ratio of about 85:15 (w/w) and was applied to form detection layer 198. As may be seen from FIG. 17, inclusion of water may increase film quality in some examples. Inclusion of water may reduce evaporation rate and result in increased film quality in some examples. In some examples, films with macroscopic cracks will produce short-circuited devices.

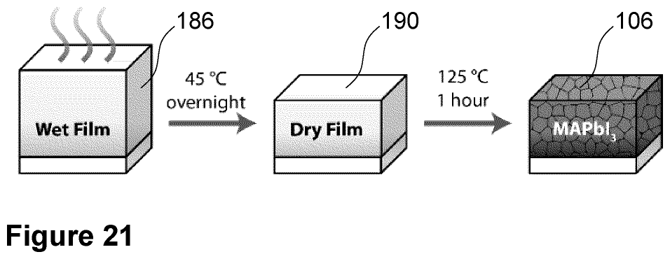

Referring now to FIGS. 20 and 21, an ink 148, such as a solution-based or pigment-based ink, may be applied using a slot die head 164 to form a precursor film 186 on a substrate 150. A vacuum chuck 188 may be used to hold the substrate 150 in position during a printing operation. Once a precursor film 186 is applied, in some example methods it may be dried for at least 12 hours at 45 degrees Celsius to produce a dry film 190, and the dry film may be further dried at 125 degrees Celsius for an hour to produce a detection layer 106. In some examples, other coating methods may be used and/or drying temperatures and times could be varied quite widely.

### EXAMPLES

The following non-limiting examples are illustrative of the present application:

### Example 1—Device Fabrication (Blade-Coating of Pigment-Based Ink)

Substrate Preparation

ITO-coated glass substrates were cleaned by sonicating for 20 min in each of detergent (extran 300, 2%), deionized water, acetone and isopropanol. The ITO substrates were stored in isopropanol until the next step.

Electron Blocking Layer Deposition

After blow-dried by nitrogen, the ITO substrates were treated by a 15 min ultraviolet-ozone immediately. Then the PEDOT:PSS film (poly(3,4-ethylenedioxythiophene) polystyrene sulfonate), using the comercialized precursor, was deposited on the ITO substrate by spin-coating with 500 rpm for 9 s and following 4000 rpm for 60 s. Next, the PEDOT:PSS covered ITO substrates were annealed at 140° C. for 20 min.

PEDOT:PSS
ITO

X-Ray Photoconductor Deposition

The smooth MAPbI₃ perovskite pigment-based ink (6 M) was prepared by mixing MAI, PbI₂ and PVB (159:461:10 weight ratio) in mixed solvent (15% wt H₂O in ethanol) with 3 hours sonication. Perovskite layers were deposited by blade-coating method with a speed of 50 mm/s after dropping the ink on the substrate. Afterwards, the fresh samples were dried at 35-40° C. for 30 mins and then annealed at 120° C. for 60 mins.

MAPbI₃
PEDOT:PSS
ITO

Dielectric Layer Deposition

After cooling down, the samples were covered by the PMMA layer using spin-coating or blade-coating method to achieve desired thickness (˜32 μm). Then the samples were put into a desiccator for drying overnight. The PMMA precursor was prepared by dissolving PMMA in chlorobenzene with 330 mg/m L.

PMMA
MAPbI₃
PEDOT:PSS
ITO

Top Electrode Deposition

To complete the device stack, Ag (70 nm) layers were sequentially deposited by thermal evaporation at a base pressure of 1×10⁻⁶ mbar.

Ag
PMMA
MAPbI₃
PEDOT:PSS
ITO

Slot-Die Coating of Solution-Based Ink

Materials and general procedures: The perovskite and interlayers were coated using a compact sheet coater (FOM Technologies) modified to Canadian electrical standards. The shim width and thickness were 13 mm×50 μm, respectively. Slot-die head alignment and gap height was set by eye with an estimated accuracy of ±10 μm. 1×3″ patterned ITO-coated glass substrates (Rs=20 Ω/sq, Xin Yan Technology Ltd.) were cleaned by sequential sonication in Extran 300 detergent (2% v/v in Millipore H₂O), Millipore H₂O, acetone, and isopropanol for 20 min each, blown dry with filtered nitrogen (0.45 μm PTFE syringe filter), dried for 5 minutes on a hot plate at 120° C. and then UV/ozone cleaned for 15 minutes prior to use. All solvents were stored over dry sieves and syringe filtered (0.45 μm PTFE syringe filter) prior to use. Methyl ammonium iodide was prepared based on previously reported procedures (Stamplecoskie, K. G., Manser, J. S. & Kamat, P. V. Dual nature of the excited state in organic-inorganic lead halide perovskites. Energy Environ. Sci. 8, 208-215 (2014)). EP-PDI was prepared using previously reported procedures (Hendsbee, A. D. et al. Synthesis, Self-Assembly, and Solar Cell Performance of N-Annulated Perylene Diimide Non-Fullerene Acceptors. Chem. Mater. 28, 7098-7109 (2016)). All other reagents were purchased from commercial suppliers and used as received.

Device Fabrication

In a 3-dram vial: PbI₂ (3.80 g, 8.25 mmol), Methyl ammonium iodide (1.31 g, 8.24 mmol), polyvinylpyrrolidone (0.256 g), cetrimonium bromide (0.102 g), and dry 9:8 NMP/DMF (2.5 mL) were combined. The mixture was stirred at 110° C. until dissolved. During this time the 1×3″ patterned ITO substrates were cleaned using the general procedure, masked using Kapton tape, and transferred to a <40% relative humidity box enclosure and carefully aligned in series. The perovskite pre-cursor was then loaded into a 3 mL polypropylene syringe and held at 95° C. for 5 minutes prior to coating. The substrates were then slot-die coated with a web speed of 50 cm min⁻¹, flow rate of 1.5 mL min⁻¹, gap height of 350 μm, and a slot-die head temperature of 100° C. Immediately after coating, the substrates are transferred to a 45° C. hot plate and dried overnight. Films were then annealed at 125° C. for 60 minutes. During this time EP-PDI (40 mg) was dissolved in 3 mL of chlorobenzene at 60° C. EP-PDI was then slot-die coated with a web-speed of 50 cm min⁻¹, flow rate of 0.4 mL min⁻¹, gap height of 250 μm, and a slot-die head/syringe temperature of 60° C. The films were then dried at 45° C. for 2 hours. The Kapton tape is then carefully removed and a small section of the perovskite was scraped off prior to bottom contact electrode deposition. Silver (100 nm) was thermally evaporated using a base pressure of 1×10⁻⁶ mbar and masked to create six 1 cm² devices per substrate. Devices did not show any immediate signs of degradation. Prior to device measurement, PMMA (50 mg/mL in CB) was then slot-die coated overtop of the silver electrodes with a web speed of 50 cm min⁻¹, flow rate of 0.6 mL min⁻¹, gap height of 250 μm, and a room temperature slot-die head. A small section of each electrode was left exposed for device measurement.

What has been described above has been intended to be illustrative of the invention and non-limiting and it will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

1. A solution-based ink formulation comprising:
a. one or more perovskite precursors;
b. a solvent or mixture of solvents;
c. a binder;
d. an additive.

2. The solution-based ink formulation of claim 1, wherein the perovskite precursor is a methylammonium perovskite of chemical formula MAPbI₃.

3. The solution-based ink formulation of claim 1, wherein the solvent is n-methyl pyrrolidone (NMP) or dimethyl formamide (DMF), or a combination thereof.

4. The solution-based ink formulation of claim 3, wherein solvent is a mixture of n-methyl pyrrolidone (NMP) or dimethyl formamide (DMF).

5. The solution-based ink formulation of claim 4, wherein the solvent is a mixture of n-methyl pyrrolidone (NMP) or dimethyl formamide (DMF) in a ratio of between about 1:2 to about 2:1 (w/w) (NMP:DMF), or about 1:1.

6. The solution-based ink formulation of claim 1, wherein the binder is polyvinyl pyrrolidone (PVP).

7. The solution-based ink formulation of claim 6, wherein the PVP has the structure: 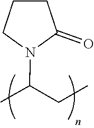
where n is an integer between 2 and 200,000.

8. The solution-based ink formulation of claim 1, wherein the additive is a quarternary ammonium salt.

9. The solution-based ink formulation of claim 8, wherein the quarternary ammonium salt is cetyltrimethyl ammonium bromide (CTAB).

10. A pigment-based ink formulation comprising
a. perovskite precursor;
b. a binder; and
c. a solvent.

11. The pigment-based ink formulation of claim 10, wherein the perovskite precursor is a methylammonium perovskite of chemical formula MAPbI₃.

12. The pigment-based ink formulation of claim 10, wherein the binder is a polymer having the structural formula 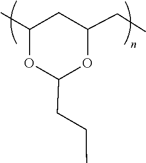
wherein n is an integer between 2 and 200,000.

13. The pigment-based ink formulation of claim 10, wherein the solvent is ethanol, water or a combination thereof.

14. The pigment-based ink formulation of claim 13, wherein the solvent is a combination of ethanol and water.

15. The pigment-based ink formulation of claim 14, wherein the solvent is a combination of ethanol and water present in a ratio of about 85:15.

16. A direct conversion x-ray detection apparatus, comprising:
a planar x-ray detection layer having a detection layer upper surface and a detection layer lower surface, the planar x-ray detection layer including a lead halide perovskite material;
a top electrode layer above the detection layer upper surface;
a bottom electrode layer below the detection layer lower surface and in conductive communication with the top electrode layer through the x-ray detection layer to apply a bias voltage across the x-ray detection layer; and
a blocking layer between the x-ray detection layer and the top electrode layer to inhibit a dark current, the blocking layer including a polymer selected from the group comprising polyacrylates, polyimides, polyamides, polysulfones, polystyrenes, and polycarbonates.

17. The x-ray detection apparatus of claim 16, wherein the planar x-ray detection layer has a thickness between the detection layer upper surface and the detection layer lower surface of at least 100 micrometers.

18. The x-ray detection apparatus of claim 17, wherein the thickness is between 400 micrometers and 1500 micrometers.

19. The x-ray detection apparatus of claim 1, wherein the blocking layer includes poly(methyl methacrylate).

20. The x-ray detection apparatus of claim 1, wherein the lead halide perovskite material is MAPbI₃.

21. The x-ray detection apparatus of claim 1, wherein the bottom electrode includes a thin-film transistor array.

22. The x-ray detection apparatus of claim 1, wherein the bottom electrode includes a transparent conducting material.

23. The x-ray detection apparatus of claim 22, wherein the transparent conducting material includes at least one of indium tin oxide (ITO) and fluorine doped tin oxide (FTO).

24. The x-ray detection apparatus of claim 1, wherein the top electrode includes at least one of silver and gold.

25. The x-ray detection apparatus of claim 1, wherein the blocking layer includes a hole transport layer inhibiting electron transport.

26. The x-ray detection apparatus of claim 1, further comprising an electron transport layer between the bottom electrode and the planar x-ray detection layer, the electron transport layer inhibiting electron hole transport.
